(19) United States
(12) Patent Application Publication
(10) Pub. No.: US 2003/0014074 A1
(43) Pub. Date: Jan. 16, 2003

(54) Method of destroying formations in a body

(76) Inventors: Daniel Cherfas, Brooklyn, NY (US); Marika Cherfas, Brooklyn, NY (US)

Correspondence Address: Ilya Zborovsky, 6 Schoolhouse Way, Dix Hills, NY, 11746, US

(21) Appl. No.: 09/902,032

(22) Filed: Jul. 11, 2001

(51) Int. Cl. A61B 17/28

(52) U.S. Cl. 606/205; 606/207

(57) ABSTRACT

A method of destroying formations in a body has the steps of introducing tubular element into a body to an area of a formation, grasping a formation by a gripping element extending through the tubular element and having a front end provided with gripping means, and destroying a formation by a formation destroying element also extending through the tubular element and having a front end provided with a working part, by rotating of the destroying element so that during rotation of the formation destroying element with the working part the part in contact with the formation destroys the formation.

### BACKGROUND OF THE INVENTION

The present invention relates to a method of destroying formations in a body, in particular in internal organs, in deep wounds and other areas which are difficult to access, without surgical operations, with minimal trauma, so that corresponding formations can be destroyed and if necessary extracted from the body. Foreign formation can include stones, growths, bullets logged in wounds, food stuck during eating, etc.

Methods of destroying corresponding formations in the body are known. Some of them include surgical methods, in accordance with which a corresponding organ is made accessible by a surgical operation, and the formation is destroyed and/or removed. Other methods include for example ultrasound crashing of stones, etc. It is believed that existing methods and devices can be further improved.

### SUMMARY OF THE INVENTION

Accordingly, it is an object of the present invention to provide method of destroying foreign formations in a body.

In keeping with these objects and with others which will become apparent hereinafter, one feature of present invention resides, briefly stated, in a method of destroying foreign or undesirable formations in a body which includes introducing into an area of a formation a mechanical rotary tool through a tubular element, bringing a working part of the mechanical rotary tool into contact with the formation, and rotating the mechanical rotary tool so as to destroy the formation by the working part of the tool.

When the method is performed in accordance with the present invention, it makes possible a destruction and if necessary withdrawal of formations from the body in a simple, efficient, and non intrusive way.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a general view of a device which uses a new method for destroying foreign formations in a body;

FIGS. 2, 3, 4, 5 and 6 are views showing a cross-section of the device shown in FIG. 1 taken along the lines 2-2, 3-3, 4-4, 5-5 and 6-6 correspondingly;

FIG. 7 is a view substantially corresponding to the view of FIG. 1 but showing a longitudinal cross-section of the device which uses a new method in accordance with the present invention;

FIG. 8 is a view showing one embodiment of the device of FIG. 1;

FIGS. 9 and 10 are views showing another embodiment of the device in accordance with the present invention in a closed and an open position correspondingly;

FIGS. 11 and 12 are views showing details of the inventive device of the embodiment of FIG. 8 in a front area;

FIGS. 13 is a view showing details of a rear area of the device which uses the inventive method;

FIG. 14 is a view showing a working head part of the device which uses the inventive method; and

FIGS. 15, 16, and 17 are views showing the device which uses the inventive method used for destruction of formations in a kidney, in a male bladder and in a food pipe correspondingly.

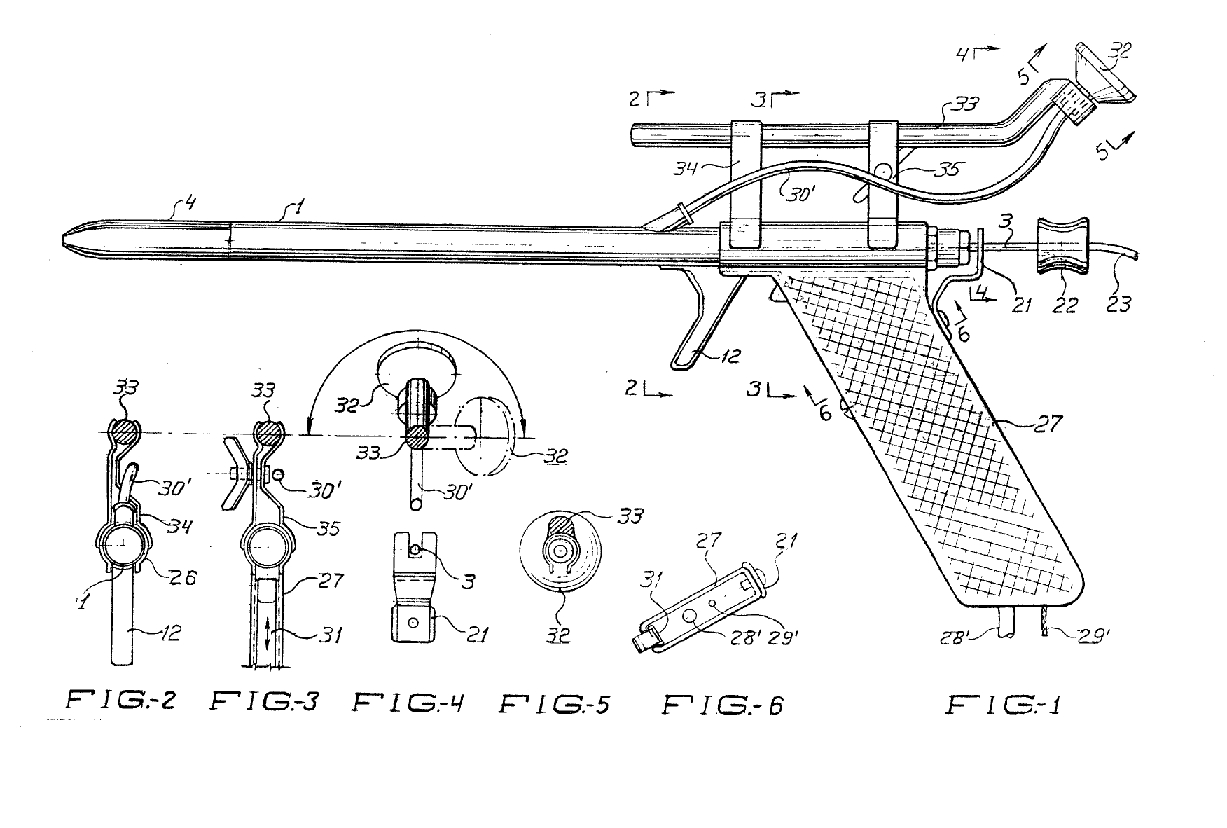

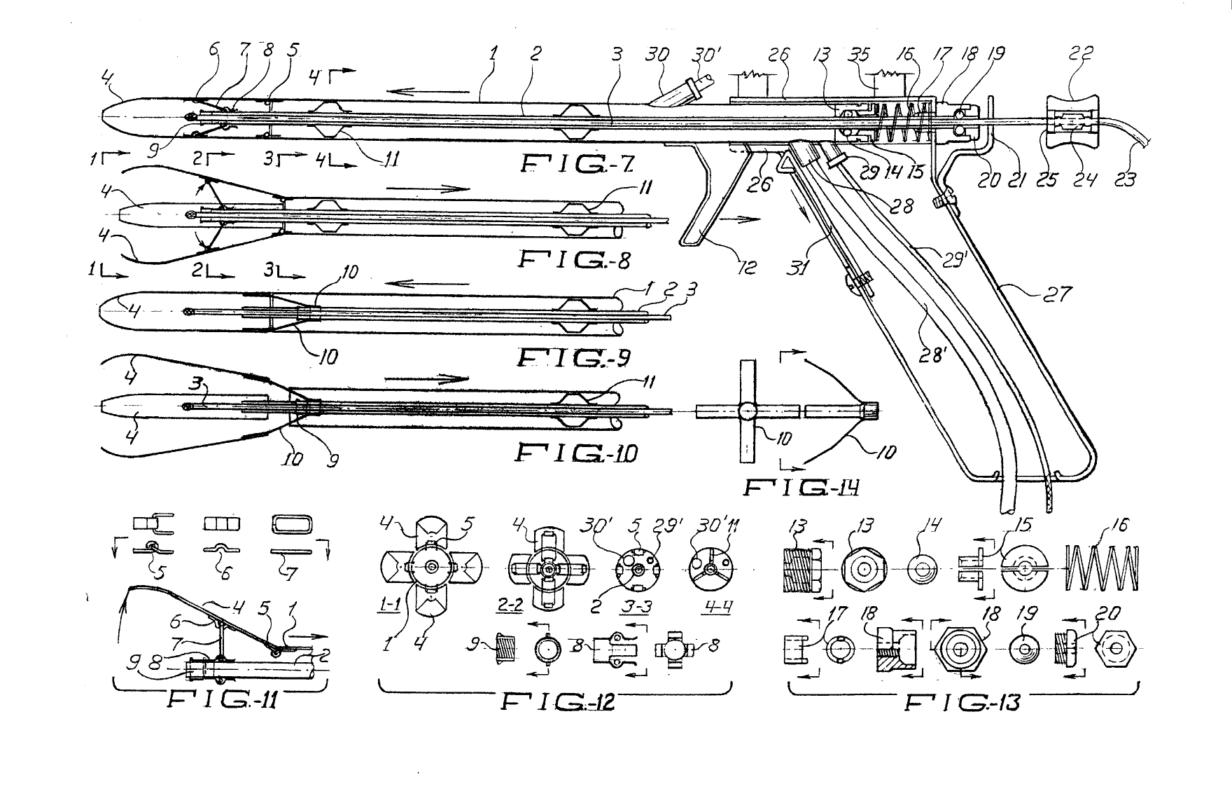

### DESCRIPTION OF THE PREFERRED EMBODIMENTS

A method of for destroying and/or extracting foreign formations in a body includes introduction of a tubular member which is identified as a whole with reference numeral 1. A tube 2 of a smaller diameter is located in the tubular member 1, and a rod 3 extends through an interior hollow of the tube 2. The rod 3 is a rigid shaft which has a front end provided with a working head, such as for example a drill, a bit, and the like. The opposite end of the rod 3 extends outwardly from the tubular member to be connected with a flexible shaft 23. The front, head part of the tubular member ends in cigar-shaped and composed of petal-shaped tongues 4. The tongues 4 have bent ends which form grippers when they are open.

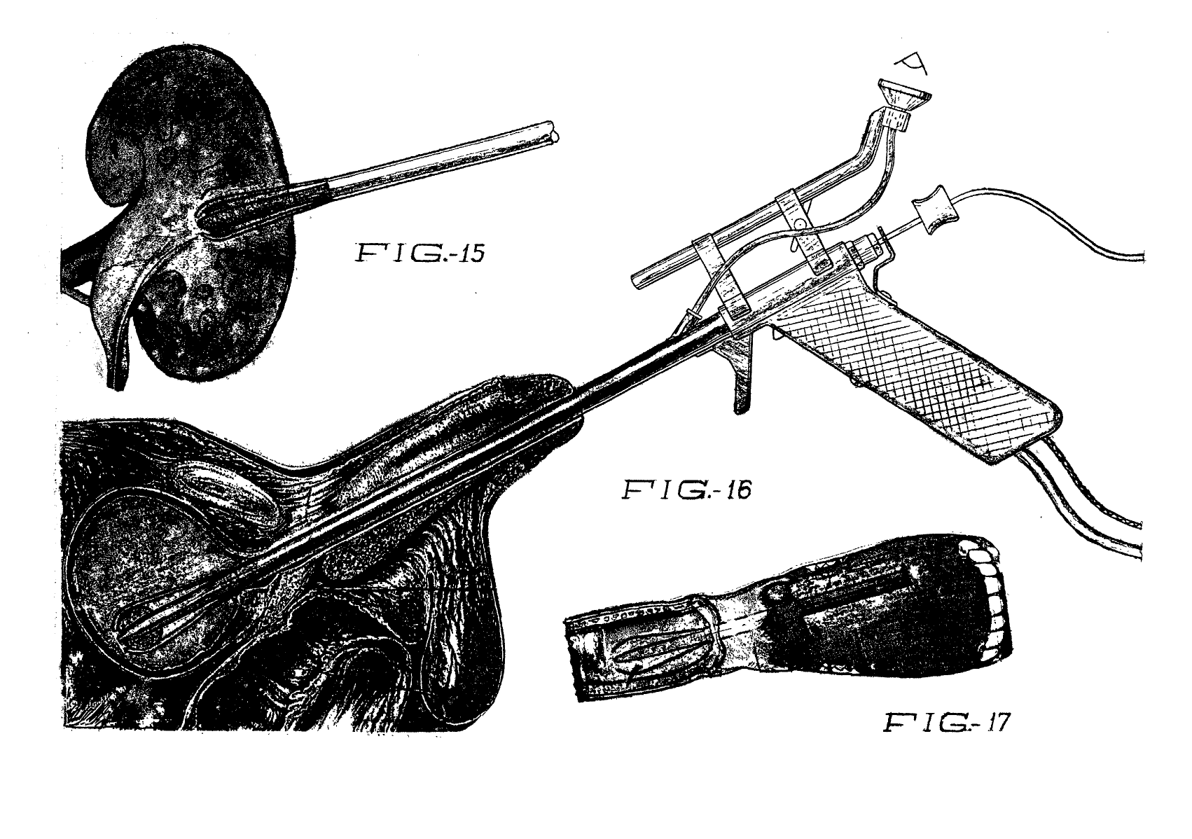

In accordance with one embodiment of the present invention which involves the use of a hinges, each tongue is provided at its base with a hinge 5 for turning. The hinge 5 includes a plate which is bent from one side and in which a hook is introduced. The plate is soldered to the tongue and the hook is soldered to the inner surface of the end of the tubular member 1 as shown in FIGS. 7, 8, 11, 12. In order to control the turning, a hinge 6 is soldered or welded to an inner surface of each tongue at a distance from its axis of turning. The hinge 6 includes a bent plate 6 with a frame 7 in it. The central element of the hinge unit is a casing 8 which is composed of a spring, alloyed steel. Bent legs of the casing embrace the tube 2 from all sides. During mounting, second ends of the frames 7 are introduced into the hooks. The casing 8 is placed on the tube 2 and fixed from one side by a soldered abutments and from another side by a bush 9 which is screwed on the end of the pipe 2. In order to turn the bush, two abutments are soldered on its outer surface.

In accordance with another embodiment of the present invention, a gripper 10 composed for example of spring, alloyed steel, is arranged on the end of the pipe 2. The gripper which is fixed by the soldered abutments on the pipe 2 from one side is also locked by screwing of the bush 9 on the end of the pipe 2. Sliders 11 are soldered on the pipe 2 and form inner supports which fix the central position of the pipe 2 in the tubular member 1.

The rear end of the tubular member is shown in FIGS. 7 and 13. In order to provide hermetization of the tubular member a special bushing 13 is screwed into its rear part from outside. The bushing has a cavity, through which the pipe 2 extends. For hermetization of the pipe 2, a sealing ring 14 is arranged on it inside the bushing. Then halves of an insert 15 which is separated into halves is introduced into the interior of the bushing 13. The outer ends of the halves of the insert are supported on the inner side of the bushing and take a pressure from a spring 16. Furthermore, for fixing the tube 2 from a longitudinal and turning movement, a special bush 17 is placed on it and located in a chamber of the spring. The left side of the bush is soldered to the pipe 2. The right side of the bush is formed with two thorns which during mounting are inserted into the drilled openings in the rear wall of the upper part of the device. A special bushing 18 is screwed on the end of the pipe 2 which extends beyond the rear wall, and is tightened. The rigid shaft 3 extends through the bushing. For the hermetization in the bushing, a sealing ring 19 is provided. The interior of the bushing 18 is closed by a plug 20 which is screwed into it and the shaft 3 freely extends through the plug. The shaft 3 extends through a limiting member 21 which is screwed to the upper part of a handle of the device.

A control handle 22 formed as a coil is arranged at the outside, rear side of the device at a certain distance. An end of the rigid shaft 3 extends from the left side into the handle, while the end of the flexible shaft 23 which extends from a rotary drive is introduced from the right end. The ends of both shafts are arranged in known rolling or sliding bearings 25. Furthermore, the ends of both shafts are introduced into a connecting coupling 24. After the connection a coil 22 is placed on the bearings and the coupling 24. After this both outside races of the bearings are fixed in the coil.

The rear end of the tubular member and the spring are located in a cylindrical element 26 which embraces them but is open in the lower part. At the front, the open part of the cylindrical member has bend parts, in which an upper part of a cock 12 can move. The rear part of the open end of the cylindrical member transits into a hollow handle of the device 27. Two nipples 28 are soldered into the lower part of the tubular member. They are used for connection of a hose 28′ for supply a cleaning medium, for example water. A nipple 29 is used for introducing an electric cable 29′ for power supply to an illuminating bulb at the end of a light guide 30′. The hose and the electrical cable are extended from the device through a lower part of the handle so as not to interfere with one another and lead to a power source.

A window which is closed by a special cover 31 is located in the upper front part of the device handle. The cover has a triple function. From the outer side, its upper bend is a limiting member for the cock 12 when it is moved rearwardly. From the inner side, the upper part of the cover blocks the spring-biased tubular member by a support for the nipple 28 in the tubular member. For disassembly of the device, the clamping screw is released and the cover is lowered so that the tubular member with the nipples can be removed and withdrawn from the window in the handle (hose 28′ and cable 29′).

The device can have a support for an eyepiece 32 and a light guide 30′ which extends from the eyepiece and has a known construction. The light guide is inserted into the tubular member through a nipple 30. The eyepiece is arranged on a standing part of a member 33. The member 33 is movable and can move forwardly and rearwardly and turn to the left and right due to its circular cross-section. This member is arranged on two supports 34 and 35 which are soldered to the casing 26. The support 35 has a tightening nut for fixing the member with the eyepiece at any lower position.

The central rod 3 or the shaft can have any length. It can be exchangeable. It can have a head with any working elements, attachments, etc.

The method is performed in the following manner.

A medical practitioner holds the device by taking its handle with all fingers of left or right hand. The tubular element is introduced into an interior organ or cavity of a patient. With monitoring of the movement of the tubular member on a screen or an X-ray device, the tubular element to a formation, for example a stone which has to be removed. With the pointing finger the practitioner pulls the cock 12, the cock presses the spring 16, so that the tubular member is moved rearwardly a few millimeters. In the embodiment with the hinge, the tubular element moves the tongues 4, the tongues 4 pull the frames 7 in which they are hingedly connected to them, and the frames 7 which have a hinged, immovable support on the tube 2 turn to the sides and move tongues outwardly. In the gripper embodiment, the gripper moves rearwardly and releases the spring biased elements of the gripper and they turn the tongues 4, so that the spaced tongues form gripping elements. The tubular element is moved forwardly, and the tongues are placed on the formation, for example on a stone. The cock is then released forwardly, and the tubular member moved forwardly. Ir the hinged embodiment, the tongues which are pushed by the tubular element turn the frames 7, and the latter are turned in the hinges of the casing 8 so as to press the tongues to the center. In the gripper embodiment, the gripping tongues which are pushed by the tubular member also will press the tongues to the center so as to form gripping members which block the stone.

Then a not shown rotary drive is energized, and the rigid drill shaft 3 is rotated together with the working head formed for example as a tool. The practitioner looks into the eyepiece and sees the stone. By displacement of the coil 22 forwardly, the practitioner introduces the drill into the stone and causes its destruction. The removal of the destroyed stone does not cause any problem.

When the method is utilized in therapeutic purposes, the end of the shaft 3 can be provided with special attachments which are located in the front area of the tubular element when it is introduced into an organ. Then the front part is opened and the shaft of the attachment is moved forwardly for treatment of corresponding problems or for administration of medications. The head of the light guide can be also moved forwardly. For this purpose, the sealing cover is released on the nipple 30 and the light guide is moved and lengthened.

With the inventive method it is possible to spread tissues in order to perform examination and touch an object which has to be destroyed, removed or treated. It is possible to destroy and remove foreign formations in internal organs, such as stones and growths of any size. It is possible to withdraw from a body foreign objects which accidently were introduced into the body. It is also possible to provide local therapeutic treatment in the interior organs, by administering corresponding medications.

It will be understood that each of the elements described above, or two or more together, may also find a useful application in other types of constructions differing from the types described above.

While the invention has been illustrated and described as embodied in method of and device for destroying foreign formations in a body, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

1. A method of destroying formations in a body, comprising the steps of introducing tubular element into a body to an area of a formation; grasping a formation by a gripping element extending through said tubular element and having a front end provided with gripping means; and destroying a formation by a formation destroying element also extending through said tubular element and having a front end provided with a working part, by rotating of the destroying element so that during rotation of said formation destroying element with said working part said part in contact with the formation destroys the formation.

2. A method as defined in claim 1; and further comprising introducing said gripping element in an interior of said tubular element.

3. A method as defined in claim 2; and further comprising forming said formation destroying element is formed as a rod which extends in an interior of said tube and also inside said tubular element.

4. A method as defined in claim 1; and further comprising illuminating an area of the formation and viewing the area through an eyepiece, and a light guide extending from the eyepiece through the tubular element.

5. A method as defined in claim 1; and further comprising for displacing the tubular element so as to release the grippers of the gripping element for grasping an object, and thereafter to moving the tubular element in opposite direction so as to cause the grippers to approach one another and to grasp the object.
